(19) United States
(12) Patent Application Publication
(10) Pub. No.: US 2006/0234934 A1
(43) Pub. Date: Oct. 19, 2006

(54) Composition and Method for Selective Cytostasis

(76) Inventor: Joseph A. Kilmon, San Marcos, TX (US)

Correspondence Address: CONLEY ROSE, P.C., P. O. BOX 3267, HOUSTON, TX, 77253-3267, US

(21) Appl. No.: 11/279,963

(22) Filed: Apr. 17, 2006

Related U.S. Application Data
(60) Provisional application No. 60/672,179, filed on Apr. 15, 2005.

(51) Int. Cl. A61K 38/52; A61K 39/42; A61K 9/127

(52) U.S. Cl. 514/12; 514/13; 424/160.1; 424/450

(57) ABSTRACT

A method of inducing cytostasis in a population of HIV-infected cells is disclosed which comprises applying to a population of HIV-infected cells a cytostatically effective amount of an inhibitor comprising an isolated peptide having the amino acid sequence FCRFLLCPSRTSD or SQCEQEGGRCRFLLCPSRTSNIGKLGCEPLWKC CKRWGG, or a conservative variant thereof, whereby cell growth in at least a portion of the HIV-infected cells is arrested, rendering the growth-arrested cells non-HIV producing. The peptide may be conjugated with an agent that is capable of selectively targeting HIV-infected cells.

### CROSS-REFERENCE TO RELATED APPLICATIONS

This application claims the benefit under 35 U.S.C. § 119(e) of U.S Provisional Patent Application No. 60/672,179 filed Apr. 15, 2005, the disclosure of which is hereby incorporated herein by reference.

### BACKGROUND OF THE INVENTION

1. Technical Field of the Invention

The present invention generally relates to the inhibition of cell growth by peptides derived from or based on amino acid sequences found in snake venom. More specifically, the invention pertains to selective induction of cytostasis by contacting a target HIV-infected cell with the peptide, or a conjugate thereof, such that cell growth and HIV production are selectively inhibited.

2. Description of Related Art

Peptide molecules with potential therapeutic value have been identified in snake venom extracts. Routine centrifugation of pooled snake venoms reveals significant yields of sloughed secretory epithelia. This cell loss results from the fact that synthesis and secretion of venom on the cellular level is a very high energy absorbing process. Gennaro, J. F., Anatomical Records, vol. 136, p. 196 (1960). The secretory epithelia therefore “burn out” at a very rapid rate and require proliferative replacement at rates in excess of normal cell lines. This excess proliferation is believed to be stimulated by a snake venom growth factor (SVGF) that exists in the venom. Federation Proceedings, vol. 40:6 (1981). One of the most potent growth factors known is Epidermal Growth Factor (EGF) that stimulates the incorporation of ³[H]-thymidine into the newly synthesized DNA of human embryonic palatal mesenchymal cells by a little more than twofold. Yoneda, T. and Pratt, R. M., Science, vol. 213, pp. 563-565 (1981). Since the snake venom growth factor (SVGF) mentioned above has been observed to increase the incorporation of ³[H]-thymidine into the DNA of Vero cells by eleven fold, SVGF is believed to be a more potent growth factor than FGF of EGF.

Other substances in snake venom with potential for therapeutic use have been described. For instance, U.S. Pat. No. 6,613,745 describes certain amino acid sequences derived from or based on snake venom toxins that have analgesic properties. U.S. Pat. No. 6,555,109 describes a non-toxic fraction isolated from the venom of Vipera xanthina that has an analgesic effect. U.S. Pat. No. 5,565,431 describes certain cancer cell inhibitors that are isolated from the venoms of poisonous snakes Crotalus atrox and Naja n. kaouthia. Those anti-cancer agents are identified as stable protein components of venoms consisting of peptides whose molecular weights are approximately 35,000 and 6,000 daltons. U.S. Pat. Nos. 4,774,31 8 and 4,731,439 describe certain low molecular weight peptides derived from Crotalus atrox venom that are said to inhibit cell growth. In U.S. Pat. No, 4,672,107, the present inventor discloses certain cytostatic snake venom fractions and purified peptides that are substantially free of cytotoxins This non-toxic fraction in snake venom is believed to function as a homeostatic regulator that returns the proliferative secretory epithelial snake cells to quiescence and to play a role in the differentiation and dedifferentiation process for epithelial secretion of venom products. Methods are disclosed by which the cytostatic fraction is substantially separated from toxic substances present in the venom, and the separated fraction exhibits substantial utility as a cell growth inhibitor.

Among the various treatment modalities for HIV/AIDS, conventional methods for challenging HIV in vivo primarily involve direct targeting of the virus itself. Some of those methods employ nucleoside-analog reverse transcriptase inhibitors (NRTI). Drugs that inhibit viral RNA-dependent DNA polyymerase (reverse transcriptase) and are incorporated into viral DNA (they are chain-terminating drugs) include Zidovudine (AZT-ZDV, Retrovir), first approved in 1987, Didanosine (ddl, Videx), Zalcitabine (ddC, Hivid), Stavudcine (d4T, Zerit), and Lamivudine (3TC, Epivir) Some of the conventional HIV/AIDS methods employ non-nucleoside reverse transcriptase inhibitors (NNRTIs) such as Nevirapine (Viramune) and Delavirdine (Rescriptor). In contrast to NRTIs, NNRTls are not incorporated into viral DNA. Instead, they inhibit HIV replication directly by binding non-competitively to reverse transcriptase. Other conventional HIV/AIDS methods employ protease inhibitors such as Saquinavir (Invirase), first approved in 1995, Ritonavir (Norvir), Indinavir (Crixivan) and Nelfinavir (Viracept). These drugs are specific for the HIV-1 protease and competitively inhibit the enzyme, preventing the maturation of virions capable of infecting other cells. Still other treatment modalities for combating HIV/AIDS rely on blockers of CD4 binding. Those drugs prevent the binding of the viral envelope protein to the CD4 receptor of the host cell or prevents the fusion between the envelope protein and the membrane of the cell.

Over the past twenty years great effort has been put into the production of a vaccine or targeted drug for the treatment and prevention of AIDS. Scientific paradigms can often sound very complicated, primarily because the vocabulary is complicated but the principle can be simple. Viral infections have always been very difficult to treat because, unlike bacteria, viruses do not have a reproductive apparatus to attack with antibiotics. Instead they insert their genetic materials into our own cells, thereby “hijacking” the reproduction apparatus of the infected cell. Each type of virus has a preferred cell that carries a natural “portal” that the virus uses to enter the cell. In the case of HIV it is a receptor protein called CD4 and co-receptors CXCR4 and CCR5 that cells of the immune system use to signal each other for responses to infection or foreign material. The immune cells, like T-4 “helper” lymphocytes, are therefore compromised and cannot perform their function and instead proliferate new viruses. HIV, like most viruses, consists of some DNA or RNA wrapped in a membrane on the surface of which are proteins that help the virus find its targets. This is not unlike the World War II antishipping mines which, in fact, HIV resembles. All of the vaccines and therapeutic modalities that have been attempted have failed so far.

An antiviral needs to accomplish its objective without being toxic to healthy host cells or being less toxic to the patient than to the virus in a given dose. The antiviral agent must rely on a consistent sequence of subunits for its target, whether a viral gene, enzyme or protein. Herein lies the “Gordian knot” of the HIV which is highly mutable, rewriting its “machine code” almost with every replication. This causes those sections of vital proteins recognized by the immune system in response to vaccines to alter its character just enough to escape immune detection or to make substitutions in a targeted genetic code. The HIV has been successful in playing “dodge ball” with every category of therapeutic agent targeted against the virus itself at some point in its construction or reproduction process.

In summary, the conventional treatment modalities are directed against the virus in one or more of the following stages of the life cycle of HIV: (1) the binding of the virus to its host cell by way of the viral envelope protein to the CD4 receptor as well as additional receptors such as CKR5; (2) the entry of the virus into the cell and the removal of its outer coat; (3) release of single stranded viral RNA and viral enzyme Reverse Transcriptase; (4) the synthesis of a DNA copy of the viral RNA by the enzyme Reverse Transcriptase; (5) the incorporation of the DNA “provirus” into the DNA of the host cell; (6) Host cell begins its own replication process transcribing its genomic DNA as well as the proviral DNA into genomic RNA and mRNA with cellular enzymes; (7) translation of viral mRNA into viral proteins by the host cell; (8) post translational modification of viral proteins in the host cell including the synthesis of the complex oligosaccharides of the viral glycoproteins by glucosidases; (9) expression of the viral envelope protein gp120 on surface of host cell; and (10) budding of new HIV from host cell taking cellular membrane as its own. Each of these modalities carries some form of toxicity to the patient causing a wide range of side effects.

### SUMMARY OF THE INVENTION

Methods and compositions are disclosed for inducing cytostasis of HIV-infected cells, and for use in HIV/AIDS therapies and cancer treatments, by applying certain non-toxic cytostatic snake venom-derived or snake venom-based cytostatic peptides to tumor cells or HIV infected cells, or other virally infected cells or any cell to be placed in stasis for in vitro studies or for therapeutic benefit. Selective cytostasis is preferably employed to carry the non-toxic cell cycle inhibitor peptide to HIV infected cells, thereby targeting not the highly mutable virus itself, or any of its components, but instead the “cradle” in which new virions are produced and also to malignant cells for the purpose of cytostasis. The cytostatic peptides, and the present compositions and methods offer a unique approach for treating cancer and HIV/AIDS, and for eliminating malignant cells or HIV and other virus infected cells from the human system.

Cytostasis means “static cell.” It is the act of causing a cell to become static and incapable of dividing. The process of placing specific cells in a non-reproductive state is called “selective cytostasis.” Two elements will be required to use selective cytostasis as a therapeutic tool. The first element is an agent that acts upon a cell to place that cell into a permanent non-reproductive, or non-dividing state. Viruses cannot reproduce by themselves. They are virtually “genes in a box” and must insert those genes into the genetic material of host cells and thereby reproduce when the host cell divides. The peptides described herein are capable of acting as an agent that, upon contact, places cells in irreversible non-dividing states. The second element of this method is the delivery system that carries the cytostatic peptide to a selected population of cells, i.e, cell that are infected with HIV. A permanent non-dividing state is induced by the cytostatic peptide, which will prevent further viral replication.

In accordance with certain embodiments of the present invention, an isolated peptide is employed which comprises the 39 amino acid sequence ser-gln-cys-glu-gln-glu-gly-gly-phe-cys-arg-phe-leu-leu-cys-pro-ser-arg-thr-ser-asp-ile-gly-lys-leu-gly-cys-glu-pro-leu-trp-lys-cys-cys-lys-arg-trp-gly-gly. Advantageously, this peptide is capable of inducing cytostasis when a cell is exposed to the peptide, and yet is non-toxic to the cell. For the purposes of this disclosure, when referring to this peptide, the term “non-toxic” means that death of a cell exposed to the peptide does not occur immediately upon contacting the cell with the peptide. Without wishing to be limited to a particular theory to explain the mechanism of action of the peptide on the affected cell, it is proposed that the cell becomes functionally inactive, cell growth ceases, the mitotic cell growth cycle is irreversibly arrested by the peptide, and apoptosis occurs, ultimately causing the death of the cell. In certain embodiments the peptide has at least six cysteine groups and three disulfide bridges. In certain embodiments the peptide comprising a blocked amino terminus.

In certain embodiments of the present invention, a purified fraction of poisonous snake venom, venom secretory epithelia, snake saliva, or a combination of any of those, from at least one venomous or non-venomous species of solenoglyphodont, proteroglyphodont, opisthoglyphodont or aglyphodont snakes is employed to arrest growth of HIV-infected cells. This purified fraction is substantially free of any toxic substances that naturally occur in the venom, epithelia or saliva and contains one of the cytostatic peptides described above. In some embodiments the purified fraction is obtained from Crotalus atrox In some embodiments the purified fraction is prepared by a process comprising: dissolving the venom, epithelia, or saliva, or any combination thereof, in an acidic medium to form an acidic solution; heating the acidic solution to a temperature and for a period of time sufficient to denature heat sensitive substances therein; centrifuging the heated solution; recovering supernatant liquid from the centrifuged solution; dialyzing the supernatant liquid to substantially remove substances with a molecular weight below about 1000 daltons to yield a dialyzed liquid; applying the dialyzed liquid to a gel filtration chromatographic column and eluting the dialyzed liquid from the column with an acidic solvent; collecting an eluate fraction from the column comprising the 39 amino acid sequence peptide having cytostatic activity and substantially no toxicity. In certain embodiments, the method includes further purifying the collected eluate fraction by high pressure liquid chromatography.

In certain embodiments the above-described purified fraction comprises substances having a molecular weight between 2000 daltons and 15,000 daltons, preferably in the range of about 3900-4400 daltons. In certain embodiments the purified fraction comprises the 39 amino acid sequence plus no more than additional 75 amino acids. In some embodiments the purified fraction comprises a peptide precursor or oligomer of 3900-4400 daltons molecular weight containing the above-identified 39 amino acid sequence.

In still other embodiments of the present invention, a method of selective treating an HIV-infected cell is provided which employs a conjugated cytostatic peptide molecule containing any of the above-described peptides and at least one conjugating agent chosen from the group consisting of polyclonal, monoclonal and bi-specific antibodies, peptides, saccharides, ligands and receptors in which the conjugated peptide molecule is capable of selectively reacting with a targeted cell to place the targeted cell into a non-proliferating state. In some embodiments the targeted cell is a tumor cell or a virally infected cell, such as an HIV-infected cell, for example.

Still another embodiment provided in accordance with the present invention is a therapeutic treatment comprising administering to a patient suffering from HIV/AlDS a composition comprising an above-mentioned isolated peptide, purified fraction, or, conjugated molecule, together with a pharmaceutically acceptable carrier.

In accordance with certain embodiments of the present invention, a method of inducing cytostasis in a population of cells is provided which comprises applying a cytostatically effective amount of an above-mentioned isolated peptide, purified fraction conjugated molecule or composition, or an isolated peptide comprising the partial amino acid sequence phe-cys-arg-phe-leu-leu-cys-pro-ser-arg-thr-ser-asp to a population of HIV-infected cells and thereby permanently rendering the cells non-HIV producing and non-proliferating. In some embodiments the method includes obtaining an above-mentioned conjugated cytostatic peptide, and selectively targeting the conjugated cytostatic peptide to the HIV-infected cells.

In certain embodiments, a method of inducing cytostasis in a population of HIV-infected cells is provided which comprises applying to the population of HIV-infected cells a cytostatically effective amount of an inhibitor comprising an isolated peptide having the amnino acid sequence of residues 9-21 of SEQ ID NO: 1, or of SEQ ID NO: 1 or a conservative variant thereof, whereby cell growth in at least a portion of the HIV-infected cells is arrested, rendering the growth-arrested cells non-HIV producing. In some embodiments, applying the peptide to the population of HIV-infected cells further renders at least a portion of the cell growth arrested cells non-proliferating. In some embodiments, rendering the cell growth arrested cells non-proliferating comprises inducing apoptosis in the cell growth arrested cells. In some embodiments, the inhibitor comprises a peptide conjugated with a conjugating agent capable of selectively associating the conjugated peptide with an HIV-infected cell, and the step of applying the peptide to the population of HIV-infected cells comprises applying the conjugated peptide. In some embodiments, the method includes selectively targeting the conjugated peptide to the HIV-infected cell. The conjugating agent may be a polyclonal, monoclonal and bi-specific antibody, peptide, liposome, saccharide, ligand or a receptor, and the step of applying the peptide to the population of HIV-infected cells may include applying the conjugated peptide.

In certain embodiments of an above-described method the inihibitor is applied to the population of HIV-infected cells in vitro culture. In other embodiments, the inhibitor is applied to HIV-infected cells in vivo, by administration to a patient suffering from HIV/AIDS. In some embodiments, the peptide has at least six cysteine groups and three disulfide bridges, and in some embodiments, the peptide has a blocked amino terminus. The blocked amino terminus may comprise, for example, an acetyl, formyl, pyroglutamyl, carbamyl, lactyl, glyceryl or glycosyl group.

In certain embodiments, an above-described method includes selecting a peptide that has an amino acid sequence consisting essentially of no more than about 75 amino acids.

Also provided in accordance with certain embodiments of the present invention is a method of treating a patient suffering from HIV/AIDS, comprising administering to a patient suffering from HIV/AIDS cytostatically effective amount of an inhibitor comprising an isolated peptide having the amino acid sequence of residues 9-21 of SEQ ID NO: 1, or of SEQ ID NO: 1 or a conservative variant thereof, wherein the inhibitor is selectively targeted to associate with HIV-infected cells in the patient, whereby cell growth in at least a portion of the HIV-infected cells is arrested, rendering the growth-arrested cells non-HIV producing. In certain embodiments, administering the inhibitor to the patient further renders at least a portion of the cell growth arrested cells non-proliferating. In certain embodiments, the method comprises selecting an inhibitor comprising a peptide having at least six cysteine groups and three disulfide bridges. In some embodiments, the method includes selecting an inhibitor comprising a peptide having a blocked amino terminus. In some embodiments, the method includes selecting an inhibitor comprising a peptide conjugated with a conjugating agent capable of selectively associating the conjugated peptide with an HIV-infected cell, and administering the conjugated peptide to the patient. The conjugating agent may be chosen from the group consisting of polyclonal, monoclonal and bi-specific antibodies, peptides, liposomes, saccharides, ligands and receptors, for example. These and other embodiments, features and advantages of the present invention will become apparent with reference to the following description and drawings.

### BRIEF DESCRIPTION OF THE DRAWINGS

FIG. 1 is a chromatogram showing the elution pattern of a cytostatically active peptide fraction from a HPLC liquid chromatography column in accordance with a method of the present invention.

FIG. 2 is a chromatogram showing the elution pattern of fraction “Q” of FIG. 1, when reapplied and eluted from a HPLC liquid chromatography column.

FIG. 3 is a photomicrograph of A-375 malignant melanoma cells in tissue culture.

FIG. 4 is a photomicrograph of tissue culture cells similar to those of FIG. 1 after application of 2 ng/ml of the 39 amino acid sequence peptide of the present invention.

### DETAILED DESCRIPTION OF THE INVENTION

In U.S. Pat. No. 4,672,107, which is hereby incorporated herein by reference, the present inventor described certain cytostatic snake venom fractions and purified peptides that are substantially free of cytotoxins. This non-toxic fraction in snake venom is thought to function as a homeostatic regulator that returns the proliferative secretory epithelial snake cells to quiescence and to play a role in the differentiation and dedifferentiation process for epithelial secretion of venom products. Methods are disclosed by which the cytostatic fraction is substantially separated from toxic substances present in the venom, and the separated fraction exhibits substantial utility as a cell growth inhibitor.

Those observations are now extended to the use of certain cytostatic peptides obtainable from poisonous snake venom, venom secretory epithelia, snake salivas, and combinations of any of those sources, for inhibiting cell growth and viral replication in HIV-infected cells. Cytostatic peptides from venomous or non-venomous snakes of various species of solenoglyphodont, proteroglyphodont, opisthoglyphodont and aglyphodont groups and have substantial activity as a cell growth inihibitor and are substantially free of cytotoxins. The presently described cytostatic peptides preferably contain the core 13 amino acid sequence disclosed in U.S. Pat. No. 4,672,107, and more preferably contain the entire “native” 39 amino acid sequence of Ser-Gln-Cys-Glu-Gln-Glu-Gly-Gly-Phe-Cys-Arg-Phe-Leu-Leu-Cys-Pro-Ser-Arg-Thr-Ser-Asp-lle-Gly-Lys-Leu-Gly-Cys-Glu-Pro-Leu-Trp-Lys-Cys-Cys-Lys-Arg-Trp-Gly-Gly. The peptides containing the full 39 amino acid sequence will have higher activity by virtue of its total tertiary folding structure as a result of its three disulfide bridges and blocked amino terminus, thus allowing conformation with its receptor.

For the purposes of this disclosure, the terms “cytotoxic” and “cytotoxins” refer to venom components that are immediately lethal to cells. For example, although the peptides disclosed herein are evolutionarily related to myotoxins, they are not inmmediately lethal when administered to a cell. The term “cytostatic” means that the cell, although not lysed or killed, goes into a permanent non-proliferative state until natural apoptosis removes it. The following examples are offered by way of illustration, and not by way of limitation. Those skilled in the art will recognize that variations of the invention embodied in the examples can be made, especially in light of the teachings of the various references cited herein, the disclosures of which are hereby incorporated herein by reference to the extent that they provide details and description supplemental to the present disclosure.

General Methods and Materials

Venom Fractionation

The cell growth inhibitory peptide or active fraction was obtained from lyophilized whole venom of Crotalus atrox (Western Diamondback Rattlesnake). A 250 mg sample of the lyophilized whole venom was reconstituted in 10 ml 1M aqueous acetic acid at 4° C. with stirring for about 1 hour. The mixture was then centrifuged at 10,000 g's at 4° C. for 2 hours. The supernatant liquid was placed in a cellophane dialysis sack and dialyzed against 2000 ml of 1M acetic acid while continuously stirring, with dialysate replacement at 4 and 8 hours.

A gel filtration chromatographic column was prepared by first suspending polyacrylamide gel (obtained from Bio-Rad Laboratories, Inc., Richmond, Calif. under the designation Bio-Gel P100) in 1M aqueous acetic acid and degassing under vacuum. The gel was packed into a column measuring 100 cm long by 2.5 cm in diameter. The column was calibrated with 5 mg each of Bovine insulin (6,000 daltons), cytochrome C (12,500 daltons) and Bovine serum albumin (67,000 daltons), mixed together in 5 ml of 1M aqueous acetic acid, added to the column, and eluted with 1M aqueous acetic acid at 0.2 ml/min. A sample of 5 ml of the dialyzed liquid from the dialysis sack was then added to the column and eluted with 1M aqueous acetic acid at 0.2 ml/min, withl eluate monitoring at 280 nm. Fractions were collected every minute, lyophilized and numbered.

Identification of the Cytostatically Active Fractions.

Each fraction was reconstituted in 110 μl of Dulbecco's modified Eagle's medium (DMEM) with fetal calf serum (FCS) 5 vol. %, 100 I.U. penicillin, 100 μg streptomycin and 2 nmole glutamine.

Malignant melanoma A375 cells (obtained from the American Type Culture Collection, Rockville, Md.) were counted in a counter (obtained from Coulter Diagnostics, Hialeah, Fla.), and about 4,000 cells were seeded into each well of a 96 well microtiter plate (obtained from Fisher Scientific, Springfield, N.J.) with 200 I.U. penicillin, 200 82 g streptomycin and DMEM containing FCS 5 vol. %, 2 nmole glutamine, and 25 nM HEPES (hydroxyethylpiperazine-ethanesulfonic acid buffer) as needed to give a final well volume of 220 mL. Following a four-day incubation period at 36.5° C., the overlying medium of the cell cultures, except for one set designated as controls, were aspirated and replaced with 110 μL of the reconstituted venom fractions. Each culture was then pulsed with 0.5 μCi³[H]-thymidine and incubated another 24 hours.

In addition to the previously mentioned controls, three additional sets of controls were established. One 96 well set of cultures was incubated without any venom fraction added thereto to insure that the medium change did not affect the cell growth. Another 96 well set of cultures contained an additional 24 μl fresh fetal calf serum. Still another 96 well set of cultures was incubated by adding 110 μl venom fraction diluant (the DMEM/FCS/penicillin/streptomycin/glutamine mixture). All cultures, including controls, were done in triplicate.

The cell cultures were terminated and harvested with a cell harvester (obtained from Flow Laboratories, McLean, Va., under the designation Titertek®), removing the contents of each microtiter plate well onto glass fiber filter paper. The filter papers were air dried and cut into strips. The strips were placed in scintillation vials and counted in 2 ml scintillation solution (obtained from Fisher Scientific, Springfield, N.J., under the designation Scintiverse®) using a scintillation counter (Model No. LS 100 C. obtained from Beckman Instruments, Somerset, N.J.) to determine the uptake of ³[H]-thymidine into newly synthesized DNA.

The venom fraction 18 fractions past the Bovine insulin marker (6000 daltons) showed 95% less ³[H]-thymidine incorporation than controls, indicating the presence of a peptide with a molecular weight less than 6000 daltons and with substantial cytostatic activity against malignant melanoma cells. Cells so treated have substantially less pseudopodal extensions and are more spheroid than control cells, further indicating the potent cytostatic activity of the fraction and possibly a permanent transformation to normal cytomorphology. A fraction obtained just ahead of the cytochrome C marker (12,5000 daltons) and estimated to have a molecular weight of about 15,000 showed similar cytostatic activity, indicating a larger precursor peptide or an oligomer. The remaining fractions were either cytotoxic or exhibited insignificant cytostatic activity.

Purification of the Cytostatically Active Peptide.

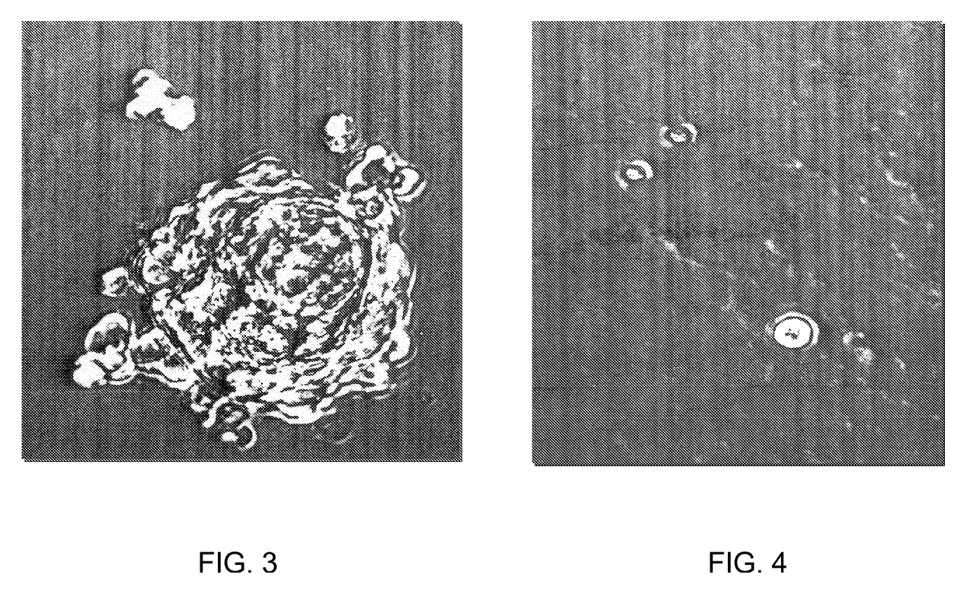

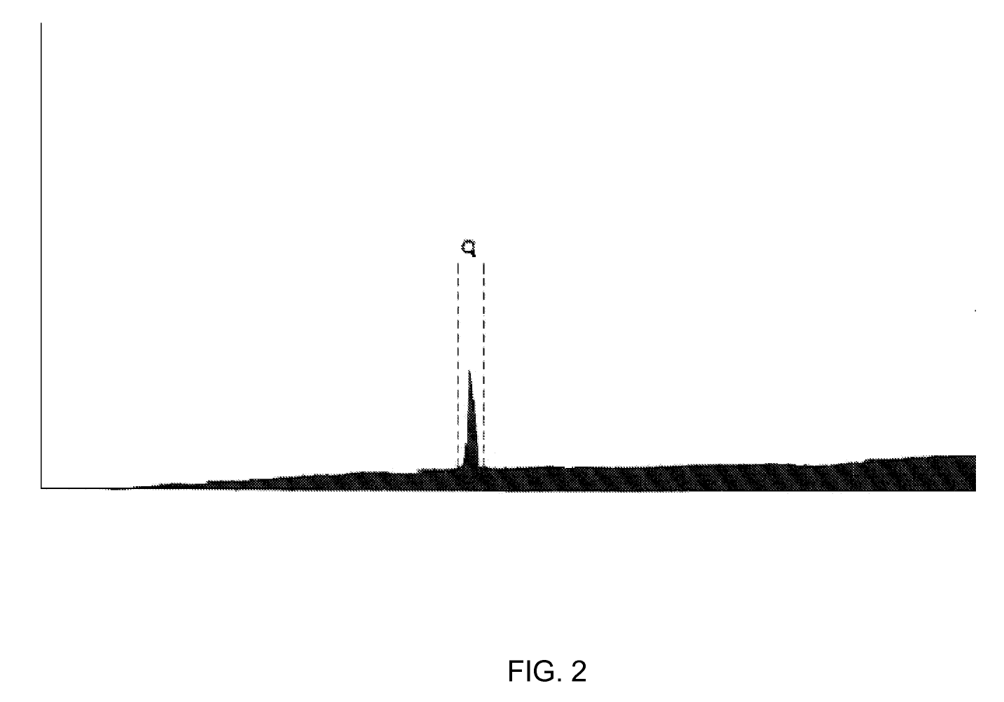

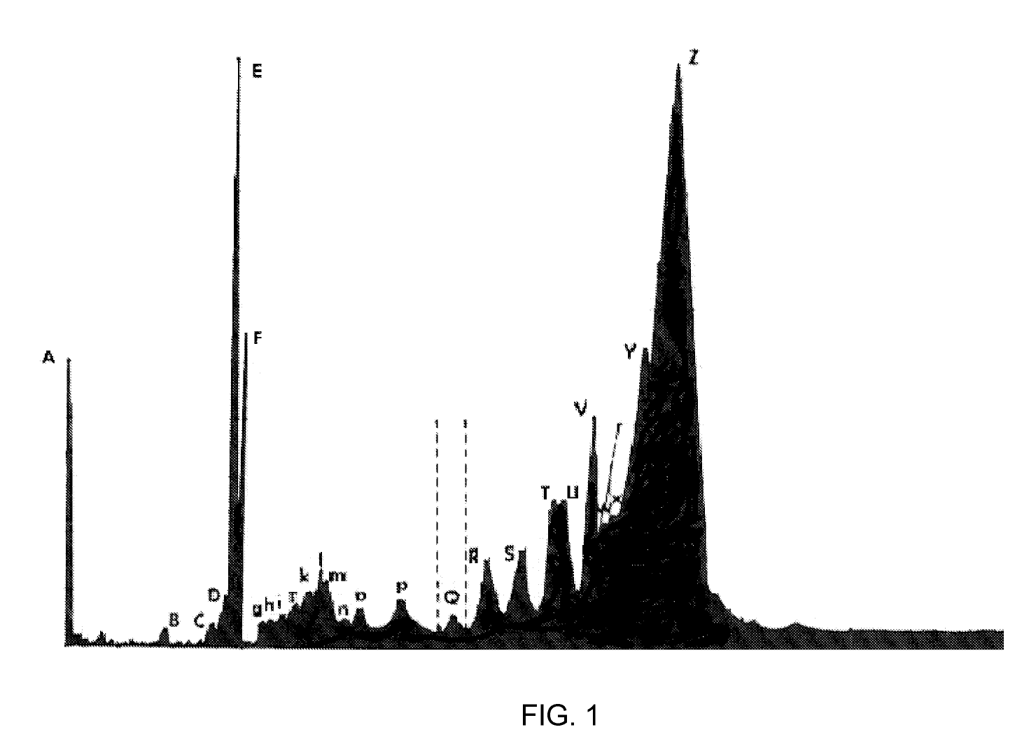

The active low molecular weight (i e., molecular weight 4,380 g/mol, extinction coefficient 12,100, volume 5300 A³) fraction was bracketed with material from adjacent fractions, pooled, lyophilized, and resuspended in 0.04 wt. % aqueous trifluoroacetic acid at a pH of 2. A 500 μl sample was injected into a high pressure liquid chromatographic (HPLC) column (obtained from Waters Associates, Houston, Tex., under the designation μ-bonde-pak C18) and eluted in 0.04 wt. % aqueous trifluoroacetic acid at 1.2 ml/min, with a linear gradient of 0-60 wt. % acetonitrile over 1 hour. The eluate was spectrophotometrically monitored at 280 nm, and fractions were collected every minute. The chromatogram is shown in FIG. 1, and 26 peaks in the range of the sample were observed. These correspond to the chromatogram peaks labeled A through Z, as indicated in FIG. 1.

The HPLC fractions were cultured with the A375 cells and assayed as described above in connection with the gel filtration chromatography fractions. The fraction corresponding to peak Q was identified as the fraction containing the cytostatically active peptide, although there was some loss of activity (about 50% less ³[H]-thymidine uptake than controls). The peak Q fraction was rerun on the HPLC to insure homogeneity, and the chromatogram is presented in FIG. 2. Additional material from the same fractions from the gel filtration was run through the HPLC column as before, and material corresponding to peak Q was collected.

Analysis of the Cytostatically Active Peptide.

The peak Q material was estimated to have a molecular weight in the vicinity of 4000 daltons by virtue of its gel filtration elution times relative to Bovine insulin. A 3 μg sample, or about 1 nmole, was analyzed for amino acid composition, and the results are seen in Table 1.

TABLE 1

| Amino Acid | nmole in 3 μg sample |
|---|---|
| Alanine | 0.5 |
| Arginine | 13.1 |
| Aspartic Acid | 3.9 |
| Cysteine | 7.9 |
| Glutamic Acid | 21.5 |
| Glycine | 20.0 |
| Isoleucine | 5.2 |
| Leucine | 14.1 |
| Lysine | 9.0 |
| Phenylalanine | 5.9 |
| Proline | 6.5 |
| Serine | 8.4 |
| Threonine | 3.0 |
| Valine | 0.4 |

The amino acid sequence of the peptide was then determined. A 4 μg sample of the peak Q fraction was digested with tosylamidophenylethyl chloromethyl ketone-treated trypsin at a substrate to enzyme weight ratio of 30:1 in 50 μl of 0.2M aqueous ammonium bicarbonate containing about 10 μg dithiothreitol for 12 hours. The digestion mixture was then applied to a HPLC column (Brownlee Aquapore C-8), and the fragments resolved in a trifluoroactetic acid/graded acetonitrile solvent system. Fragments were selected for sequence analysis with a gas phase protein sequencer (Model No. 470A, Applied Biosystems, Foster City, Calif. A 39 amino acid sequence of serine-glutamine-cysteine-glutamic acid-glutamine-glutamic acid-glycine-glycine-phenalalanine-cysteine-arginine-phenylalanine-leucine-leucine-cysteine-proline-serine-arginine-threonine-serine-aspartic acid-isoleucine-glysine-lysine-leucine-glycine-cysteine-glutamic acid-proline-leucine-tryptophan-lysine-cysteine-cysteine-lysine-arginine-tryptophan-glycine-glycine (SEQ ID NO: 1) and a blocked amino terminus was determined.

Although purification of the cytostatic peptides from snake venom is exemplified above, and is the method generally preferred by the inventor, an alternative method of producing a disclosed cytostatic peptide may be employed instead, if desired. For instance, conventional recombination techniques may be used to produce the disclosed cytostatic peptides in recombinant form, or they may be genetically engineered or modeled by any appropriate chemical synthetic process which is known in the art. One such process is the synthetic modeling of drugs based on the topology of low molecular weight protease inhibitors in snake venoms known as Angiotensin Converting Enzyme Inhibitors, such as Captopril and its many homologs.

Isolated Cytostatic Peptide.

The preferred cytostatic peptide is an isolated amino acid sequence comprising serine-glutamine-cysteine-glutamic acid-glutamine-glutamic acid-glycine-glycine-phenalalanine-cysteine-arginine-phenylalanine-leucine-leucine-cysteine-proline-serine-arginine-threonine-serine-aspartic acid-isoleucine-glycine-lysine-leucine-glycine-cysteine-glutamic acid-proline-leucine-tryptophan-lysine-cysteine-cysteine-lysine-arginine-tryptophan-glycine-glycine (SEQ ID NO: 1), which possesses cytostatic activity when it is administered to a cell in vitro or in vivo. The peptide preferably has no more than 75 amino acids, a portion of which is the above-identified 39 amino acid sequence. Variants of this 39 amino acid sequence having at least 90% sequence identity, preferably 95% sequence identity, and/or which includes as many as four additional or fewer amino acids are expected to possess similar cytostatic activity and are also considered to be a part of this disclosure.

It is also preferred that the amino terminus of the cell growth inhibitory peptide is blocked. For example, an acetyl, formyl, pyroglutamyl, carbamyl, lactyl, glyceryl or glycosyl group may be bound to the terminal amino acid. The peptides containing the full 39 amino acid sequence will have higher activity due to the total tertiary folding structure resulting from that sequence's three disulfide bridges and blocked amino terminus, thus enhancing the peptide's ability to conform with its receptor.

A pharmaceutical composition for inhibiting in vivo cell growth comprises an above-described cysteine-rich peptide having enhanced cytostatic activity and which is substantially free of cytotoxic activity, together with a pharmaceutically acceptable carrier. Suitable carriers are generally known in the art.

Alternatively, a purified fraction of venom, venom secretory epithelia, salivas or a combination of venom and epithelia, from a species of solenoglyphodont, proteroglyphodont, opisthoglyphodont, and aglyphodont groups of venomous or non-venomous snake may be used. The purified cytostatic fraction as described is substantially free of toxic substances occuring in the venom and the venom secretory epithelia, and contains an acid and heat stable peptide exhibiting substantial utility as a cell growth inhibitor. The fraction is preferably obtained by a method which includes the steps of (a) separating an acid-soluble, heat stable portion from the venom, the venom secretory epithelia, or a combination thereof, and (b) separating the peptide-containing fraction from the acid soluble portion by dialysis, gel filtration chromatography, high pressure liquid chromatography, electrophoresis, electrofocusing chromatography, ion exchange chromatography or a combination thereof.

Cytostatic Peptides and Conjugates.

A new method of inhibiting cell growth includes the step of introducing to the cells to be inhibited a peptide substantially free of cytotoxins and containing an amino acid sequence of serine-glutamine-cysteine-glutamic acid-glutamine-glutamic acid-glycine-glycine-phenalalanine-cysteine-arginine-phenylalanine-leucine-leucine-cysteine-proline-serine-arginine-threonine-serine-aspartic acid-isoleucine-glysine-lysine-leucine-glycine-cysteine-glutamic acid-proline-leucine-typtophan-lysine-cysteine-cysteine-lysine-arginine-tryptophan-glycine-glycine (SEQ ID NO: 1), or a conservatively modified variant thereof. The peptide preferably contains this sequence of 39 amino acid groups and a blocked amino terminus, and has a molecular weight of about 3900-4400 daltons. The peptide is also preferably obtained by fractionation of venom, venom secretory epithlelia, salivas or a combination of venom and the epithelia, from a species of solenoglyphodont, proteroglyphodont, opisthoglyphodont and aglyphodont groups of venomous or non-venomous snakes.

The isolated peptide preferably comprises the cysteine-rich amino acid sequence serine-glutamine-cysteine-glutamic acid-glutamine-glutamic acid-glycine-glycine-phenalalanine-cysteine-arginine-phenylalanine-leucine-leucine-cysteine-proline-serine-arginine-threonine-serine-aspartic acid-isoleucine-glysine-lysine-leucine-glycine-cysteine-glutamic acid-proline-leucine-tryptophan-lysine-cysteine-cysteine-lysine-arginine-tryptophan-glycine-glycine (SEQ ID NO: 1) For convenience, this sequence is sometimes referred to herein as “++FCRFLLCPSRTSD++”. It is believed that any peptide having the “core” or partial amino acid sequence of FCRFLLCPSRTSD, corresponding to residues 9-21 of SEQ ID NO: 1, has substantial utility as a cell growth inhibitor, regardless of whether it is synthesized or derived from natural sources. By the term “cell growth inhibitor” is meant a substance the presence of which produces a substantial cytostatic effect on cells, such as is indicated, for example, by a marked reduction of ³[H]-thymidine uptake on incubation. The peptides containing the full 39 amino acid sequence will have higher activity than the “core” peptide due to the total tertiary folding structure resulting from the larger sequence's six cysteine residues at positions 3, 10, 15, 27, 33 and 34, the three resulting disulfide bridges, and, preferably, the blocked amino terminus, thus enhancing the peptide's ability to conform with its receptor.

Preferably, the isolated peptide comprises the above-described 39 amino acid sequence and has a molecular weight of about 3900-4400 daltons, although larger peptide precursors and oligomers containing the 39 amino acid sequence are also contemplated. In addition, the peptide is preferably acid soluble and stable, and heat stable. Preferably the cytostatic peptide has a blocked amino terminus. For instance, the amino terminus may be blocked in post translational modification by acetyl, formyl, pyroglutamyl, carbamyl, lactyl, glyceryl or glycosyl groups. A glycosyl blocking group is likely in the native peptide. The cell growth inhibitor may be obtained as partially or highly purified fraction of venom, venom secretory epithelia homogenate, salivas or a combination of venom and venom secretory epithelia homogoenate, hereinafter sometimes collectively referred to as “venom,” from any species of proteroglyphodont, solenoglyphodont, opisthoglyphodont and aglyphodont groups of venomous or non-venomous snakes. Because the fraction obtained is believed to be more cytostatic and to occur in a greater relative proportion, the cell growth inhibitor is preferably obtained from the venom of a species of Sistrurus or Crotalus (rattlesnakes), particularly the species Crotalus atrox (Western Diamondback Rattlesnake) but homologs with slightly variant sequences from all other proteroglyphodont, solenoglyphodont, opisthoglyphodont and aglyphodont groups of non-venomous snakes are contemplated.

As described above in the General Methods and Materials, the cytostatically active venom fraction may be obtained by first substantially separating the acid-soluble, heat stable portion from the venom, and then substantially separating the peptide-containing fraction from the acid-soluble, heat stable portion by dialysis, gel filtration chromatography, electrophoresis, electrofocusing, high pressure liquid chromatography, ion exchange chromatography or a combination thereof, to yield a more highly purified fraction or substantially pure peptide.

The acid-soluble, heat stable portion is preferably separated from lyophilized whole venom, although fresh or fresh frozen venom may also be used. The venom is reconstituted in a suitable acidic medium, such as, for example, a solution of 0.1-10M acetic acid or the like in water or a suitable organic solvent. The insoluble portion can then be removed by filtration, centrifugation of the like, preferably by centrifugation, and the acid-soluble portion recovered.

Typically, reconstitution of 250 mg lyophilized venom in about 10 ml of 1M acetic acid in water with stirring for one hour, centrifuging the reconstituted mixture at 10,000 g's for 2 hours at 4° C., and collecting the supernatant liquid is adequate for this purpose. Optionally, the venom or the acid soluble portion thereof may be heated to a temperature (up to 100° C.) and for a period of time sufficient to substantially react heat instable substances therein.

A fraction containing the cytostatically active peptide is then separated from the acid-soluble portion by dialysis, gel filtration chromatography, electrophoresis, electrofocusing, high pressure liquid chromatography, ion exchange chromatography, or similar separation techniques, or by a combination of these techniques. Preferably, the acid-soluble portion is first dialyzed to remove very low molecular weight substances, e. g, substances with molecular weights below about 1000 daltons, and then separated into fractions substantially according to molecular weight by gel filtration chromatography. Then, if desired, the cytostatically active fraction obtained by the gel filtration chromatography may be, and preferably is, further purified by high pressure liquid chromatography.

The dialysis of the acid-soluble portion of the venom may be effected by placing the acid-soluble venom portion in a dialysis sack made of a semipermeable material and dialyzing against a suitable solvent which preferably has substantially the same constitution as the liquid medium in which the venom was reconstituted. Typically, dialysis in a cellophane dialysis sack against 1M aqueous acetic acid for a period of about 12 hours with dialysate replacement at 4 and 8 hours is adequate to substantially remove substances with molecular weights below about 1000 daltons, although similar techniques which effect about the same degree of separation are also suitable.

The dialyzed portion, i.e., the portion not passing through the semipermeable membrane, is then separated by gel filtration chromatography or a similar technique into a plurality of fractions according to relative molecular weights, Typically, the gel filtration chromatographic column is packed with polyacrylamide gel, polysaccharide gel, or the like, and a sample of the dialyzed acid-soluble venom portion is placed in the column and eluted therethrough with a carrier at a predetermined flow rate. The carrier may be any suitable solvent, such as water or an organic solvent and preferably has the same constitution as the liquid medium in which the venom was reconstituted and the acid soluble portion thereof dialyzed against. The fractions are collected as eluate at predetermined intervals, or as indicated by an eluate detector, such as, for example, a spectrophotometer, preferably monitoring at about 280 nm. Preferably, each fraction obtained by the gel filtration chromatography contains venom components with molecular weight variances of about 200-500 daltons or less. Also contemplated are similar separation techniques which provide substantially equivalent resolution of the venom into fractions according to molecular weight, such as, for example, electrophoresis, electrofocusing or the like.

If the elution time of the cytostatically active peptide is not known, each fraction of collected eluate is introduced into a culture which is then incubated, and the fraction containing the cytostatically active peptide is identified by observing the relative growth rates of the cultures. The growth rate of the cultures is conveniently determined, for example, by assaying the cultures for ³[H]-thymidine incorporation into newly formed DNA. Once the cytostatically active fraction is identified, the elution time thereof can be readily determined. Thereafter, if a new or different gel filtration column is used, or if the gel filtration conditions are changed, a sample of the previously collected fraction can be used to readily determine the elution time of the desired peptide-containing venom fraction.

With sufficient resolution by gel filtration to substantially remove cytotoxic substances, the fraction containing the cystostatically active peptide may be used in its crude form as a cell growth inhibitor, but may also be, and preferably is, further purified to substantially remove cytostatically inactive substances as well. Preferably, the cytostatically active fraction is further purified by high pressure liquid chromatography (HPLC) by elution with a suitable solvent through a suitable packed chromatograph column. For example, elution with about 0.05 wt. % aqueous trifluoroacetic acid with a gradient of acetonitrile, initially at 0 wt. % and gradually increased to about 60 wt. % of the elution solvent, through a column such as μ-bondapak C18 obtained from Waters Associates, Houston, Tex., is suitable for this purpose, although similar HPLC techniques or the like are also contemplated.

The above-mentioned cell growth inhibitor of SEQ ID NO: 1 is a particularly effective cytostatic agent. Since it is substantially free of cytotoxins, the cell growth inhibitor can be used to inhibit the growth of cells by introducing an effective amount of the cell growth inhibitor to the cells to be controlled. Cells amenable to such growth inhibition include in vitro cultured cell lines. Further, it is contemplated that the growth of cells may be inhibited in vivo in animals such as mammalia, including human, since it is substantially free of cytotoxins.

Induction of Cytostasis in HIV-Infected Cells.

Selective Cytostasis differs from all other known antiviral strategies by targeting the “hijacked” cells, rather than any component of the virus itself, with an above-described peptide or peptide conjugate. For example, the peptide corresponding to residues 9-21 of SEQ ID NO: 1 immediately places the infected cell in an irreversible non-reproducing state thereby preventing the hijacked reproductive process from making more viruses. The viral genetic material incorporated into the DNA of the infected cell does not get the opportunity to make new viruses before the infected cell is naturally eliminated by the body following its normal life-span. A peptide conjugate containing this 13 amino acid peptide will be targeted specifically to HIV infected cells taking advantage of surface proteins peculiar to infected cells. It is expected that viral load in HIV infected individuals will be markedly reduced.

A cytostatic peptide is targeted to cells infected with the HIV to place the cells in a permanent or irreversible non-dividing state and prevent the proliferation of mature virions. The peptide conjugate may be prepared using another suitable ligand or receptor. Examples of such ligands and receptors are soluble CD4 and small active site peptides of pathogenic, viral or oncogenic substrates or enzymes to be activated at the target and antigen binding sites of bi-specific antibodies, as described by Raso and Griffin (1981). Preferably, the cytostatic peptide molecule is conjugated to a humanized monoclonal antibody or bi-specific antibody molecule, according to any suitable protein-antibody conjugation technique that is known in the art and is commercially available. For example, a method similar to that described in U.S. Pat. No. 4,859,449 (Mattes) or U.S. Pat. No. 5,165,923 (Thorpe, et al.) can be employed. Preferably the cytostatic peptide to be conjugated contains the above-described 39 amino acid sequence, and preferably includes no more than about 75 additional amino acids. Alternatively, the cytostatic peptide used for conjugation contains at least the 13 amino acid sequence FCRFLLCPSRTSD (corresponding to residues 9-21 of SEQ ID NO: 1) and has a molecular weight of at least 2000 daltons, but no more than 15,000 daltons. Preferably the molecular weight of the cytostatic peptide prior to conjugation is in the range of about 3900-4400 daltons.

In some embodiments of this method, a recombinantly made peptide is conjugated to a humanized monoclonal antibody or antigen binding site of a bi-specific antibody. The recombinant peptide is then joined to one or more specific markers or epitopes chosen by their affinity ratios. Suitable recombination and conjugation procedures are well known in the art. In some embodiments, the peptide and/or conjugating agent is selected so that the conjugated or unconjugated configuration of the recombinant peptide will tender the cytostatic peptide inactive anywhere except in the cytoplasm of the targeted cell population that could include neoplastic, virally infected or pathogenic marrow or immune cells preparatory to stem cell or marrow transplantation. A population of virally infected, oncogenic or other pathologically involved cells is contacted with the conjugated peptide, in vitro or in vivo, by addition of the peptide or conjugated peptide to the cell culture medium. The peptide is transported into the cell (e.g, by endocytosis or ligand-receptor interaction and internalization). An HIV positive patient will have viral load quantified prior to treatment with cytostatic peptide conjugates, as described.

Viral load tests measure HIV RNA, which contains the instructions for making more virus. There are several different viral load tests that are known in the art, and are described in the literature. Two such tests are currently approved for general use. One of which is the Amplicor HIV-1 Monitor test, better known as the PCR test. The other is the NucliSens HIV-1 QT, or NASBA. The most sensitive of the available Viral Load Test Kits will be selected and used for all subjects.

Treatments that reduce viral load lead to improved health. Improved health will be most noticeable in people who start the studies with a vital load of over 20,000. For these people, a reduction in vital load of 70% or more for as little as 8 weeks will reduced their chance of experiencing any disease progression by half and a reduction of 90% or more will be significant. A targeted peptide or conjugated peptide comprising SEQ ID NO: 1, or a conservatively modified variant thereof that retains the cysteine bridges and amino terminal blockage, or the peptide corresponding to residues 9-21 of SEQ ID NO: 1, is expected to reduce Viral Load significantly. Especially after administration as two or more treatments set apart to coincide with the natural cell death range of T and B lymphocytes. The longest cell life CD4 bearing cell types such as Follicular Dendritic cells, mnonocytes and macrophages, may require long term monitoring and additional infusions of the targeted cytostatic peptide.

Viral Load tests will be conducted at 12 hours, 24 hours, 3 days, 1 week, 2 weeks after first treatment for a statistically significant number of the test population, and continued scheduled tests will be conducted over months, or years, to prevent activation of latent virus. It is expected that, at 12 hours post infusion, a range between an elevation of viral load to a noticeable reduction is expected, in a group of patients. At 24 hours, either a plateau effect or some reduction is expected. At 3 days, a noticeable reduction in viral load is expected, and a significant reduction is expected at 1 week. Viral load is expected to be trivial or absent at 2 weeks.

The conjugated and unconjugated peptides are expected to have therapeutic application for treatment of HIV/AIDS in symptomatic and nonsymptomatic patients suffering from HIV infection or AIDS. In vitro tests with a specific patient's donor peripheral blood mononuclear cells (PBMC) may be even more indicative than tests with HIV positive H9 or CEM cells lines from commercial sources as indicators of in vivo effectiveness of the cytostatic peptides and peptide conjugates.

“Selective Cytostasis” is a unique approach that targets the cells in which HIV proviruses reside rather than targeting various elements of the virus itself which eventually mutates after frequent replications. Selective cytostasis differs from known antiviral strategies by targeting the “hijacked” cells, rather than any component of the virus itself, with a cysteine-rich cell growth inhibitory peptide or venom extract described in U.S. Pat. No. 4,672,107 or in the present disclosure. The cytostatic peptide or venom fraction immediately places the HIV infected cell, or malignant cell, in an irreversible non-dividing state thereby preventing the replicative process from reproducing more virus or tumor cells. The viral genetic material incorporated into the DNA of an HIV infected cell does not get the opportunity to make new viruses before the infected cell is naturally eliminated by the body following its normal life-span. This is the cytostasis element of the therapeutic approach, making the infected cell “static.” This element was accomplished with certain peptides and venom fractions described in U.S. Pat. No. 4,672,107. This accomplishment is presently extended and improved, especially by employing cell selectivity methods and compositions to carry both new and previously disclosed cytostatic peptides specifically to HIV infected or malignant cells or other cellular targets. Preferably the highly active cytostatic peptides are conjugated to humanized monoclonal antibodies, bi-specific antibodies, or other ligands and receptors to conserved markers or targets on the malignant or the HIV infected cell surface, or on other cells selected for cytostasis. These immunoconjugates and ligands will make the new selective cytostasis methodologies a more effective approach to non-toxic AIDS, antiviral and cancer therapies.

An alternative way of effecting selective cytostasis in HIV-infected cells adapts the Antibody Directed Enzyme Prodrug Therapy (ADEPT) technology of Seattle Genetics. For instance, a method may employ a precursor or oligomer of the cytostatic peptide, which could be the “prodrug,” and an antibody directed enzyme to either HIV infected cells or cancer cells that would activate the cytostatically active peptide in situ. ADEPT technology for cancer therapy is a two-step approach. Initially a mAB-enzyme conjugate is administered, which localizes on the tumor cell surface and clears from the systemic circulation over time. In the second step an anticancer prodrug is administered, which circulates throughout the body and is converted to the active drug by the enzyme, which can then penetrate in to cells and exert cytotoxicity (Senter, P. D. et al. (2001); McDonagh, C F et al (2003)). ADEPT platform by Seattle Genetics utilizes the recombinant fusion protein L49-sFv-bL (SGN-17) as the mAB-enzyme conjugate and C-Mel (SGN-19) as the prodrug. L49-sFv-bL consists of a single chain variable fragment (sFv) of mAB L49 linked to a mutant form of bacterial b-lactamase (Siemers, N O et al. (1997)). L49 is a mAB that binds to p97 melanotransferrin antigen that is expressed in human melanomas and carcinomas (Siemers, N O et al. (1997)). b-lactamase catalyzes the hydrolysis of prodrug C-Mel to active drug melphalan (McDonagh, C F et al (2003))), which is an alkylating agent that is used to treat cancer.

The ADEPT system SGN-17/19 has several features that indicate its potential to become a therapy with high efficacy. Studies have shown that mAB-enzyme conjugate SGN-17 is homogeneous, localizes in solid tumor masses (Siemers, N O et al. (1997)), and tumor to blood ratio of it is more than 100 within 24 hours after administration (Wu, A M (2005)). This rapid clearance of mAB-enzyme conjugate from the systemic circulation is a critical parameter for it being suitable for therapy. Specificity is achieved by utilizing a non-human enzyme. Usually immunogenicity becomes a challenge in creating successful monoclonal antibody therapy. Despite its bacterial origin, b-lactamase indicates low immunogenicity in humans (Jung, M (2001)) ADEPT technology has several advantages over other nAB-based therapies. It can be adapted to be used with various combinations of mABs, enzymes, and prodrugs ADEPT method has the ability to use a prodrug that is converted in to a drug that is too toxic for therapeutic use if administered in its active form. Another benefit is a single molecule of mAB-enzyme is sufficient to generate a high concentration of active drug at the tumor site and since the drug is riot covalently linked to immunoconjugate, it is able to freely diffuse throughout the tumor (Senter, P. D. et al, (2001)). Since the drug is selectively generated within tumor masses, side effects are reduced compared to the systemic drug administration (Jung, M (2001)) which makes ADEPT a promising therapeutic approach.

Induction of Cytostasis in Cancer Cells.

It is proposed that the cytostatic peptides in their conjugated or unconjugated form are useful as an anti-tumor and tumoristatic agent in the treatment of a wide variety of benign and malignant neoplasms, such as carcinomas, melanomas, sarcomas, and leukemias. Generally, introduction to the cancer cells of about 50 ng of the cytostatically active peptide is sufficient to arrest the proliferations of at least about 100,000 melanoma cells. FIG. 3 is a photomicrograph of A-375 malignant melanoma cells in tissue culture. FIG. 4 is a photomicrograph of tissue culture cells similar to those of FIG. 3 after application of 2 ng/ml of the 39 amino acid sequence peptide of the present invention.

Similar results were obtained with other cancer cell lines. For example, the ALL line MOLT-4 (now denoted CRL1582 by the ATCC) was used for testing a representative cytostatic peptide for its effect on leukemic cells. The testing method described by Kikuwa, et al (1986), which is hereby incorporated herein by reference. In brief, the cells were maintained as a suspension in RPMI 1640 (Gibco) and 10% FCS, 100 u. penicillin G/mL and 100 micrograms streptomycin/mL with 2 mM L-glutamine. They were seeded 3×10⁵ into 96 well microtiter plates in the same manner as the A-375 cell, as described above and in U.S. Pat. No. 4,672,107, with 50 nG/mL of the HPLC peak Q material with 90-95% less ³[H]thymidine uptake than controls and the cells presenting an altered “rounded” visual appearance.

Representative non-toxic cytostatic peptides were tested in still other cancer cell lines. For instance, a representative cytostatic peptide fraction, in nanogram quantities, was applied to a Primary Breast Carcinoma (ATCC CRL2320), lung carcinoma (ATCC A549), the ALL line above (MOLT-4 CRL1582) and AML (ATCC CCL246), osteosarcoma (CRL1423), A431 vulvar epithelial carcinoma under the same conditions as the A-375 test and with the same result of total cytostasis. None of those in vitro test cells resumed proliferation even after washing three times and replaced in fresh medium with FCS. The non-proliferative state induced by the cytostatic peptides is irreversible. The in vitro test systems are considered to be models for what can be expected, to at least some extent, when the peptides are applied locally to similar types of cells in vivo.

The cell growth inhibitor is preferably introduced to the target cells by direct injection at the primary site, or by oral or intravenous administration if the cytostatic peptide is conjugated with a tumor-specific antibody, to induce “selective cytostasis,” as discussed above with respect to HIV/AIDS treatment. Cell stasis, and not toxicity, is a major benefit in the presently disclosed treatment of cancers. By contrast, conventional treatments for HIV or cancers that consist of toxic agents either administered intravenously or as conjugates with antibodies or other ligands can have debilitating side effects. With immunoconjugates to such toxic agents as Ricin or PAP targeted against specific cell targets there is always the danger of “errant” toxin that may cause toxic side effects. The current method employs a non-toxic peptide with profound effect on the targeted cells without toxic side effects.

Without further elaboration, it is believed that one skilled in the art can, using the description herein, utilize the present invention to its fullest extent. The foregoing embodiments are to be construed as illustrative, and not as constraining the remainder of the disclosure in any way whatsoever. While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. For example, any variation in sequence, genetic or protein, from a species of solenoglyphodont, proteroglyphodont, opisthoglyphodont or aglyphodont group of venomous or non-venomous snakes that exhibit activity and characteristics of the representative cysteine rich protein described herein is within the spirit of the invention. The 39 amino acid sequence of SEQ ID NO: 1 is expected to be an effective probe for isolating and identifying other cysteine-rich protein, polypeptide or oligomer that are active, to at least some extent, for rendering HIV-infected cells non-virus producing and non-proliferative.

Conservatively modified variants of SEQ ID NO: 1 will be apparent to those skilled in the art, and methods employing such variants and are also considered to fall within the scope of this invention. For the purposes of this disclosure, “conservatively modified variants” of SEQ ID NO: 1 include amino acid substitutions, deletions and/or additions that do not substantially affect the character of the variant peptide relative to the starting peptide (i. e., the disclosed 39-amino acid sequence of SEQ ID NO. 1). Preferably, a conservatively modified peptide consists of no more than 75 peptides, and, more preferably, has at least about 90% sequence identity to SEQ ID NO: 1. The variant peptide's character is not substantially affected if the tertiary folding structure attributable, at least in part, to the three disulfide bridges arising from cysteine residues at positions 3, 10, 15, 27, 33 and 34 of SEQ ID NO: 1, and, preferably, the blocked amino terminus of the parent peptide, are retained after substitutions, insertions, additions and/or deletions are made to the parent peptide, thus allowing conformation of the modified peptide with its receptor. For example, any substitution of aliphatic, hydrophobic, hydrophilic, charged or uncharged amino acids may be made to alter the disclosed 39 amino acid sequence, genetically or by conventional protein synthetic methods, to produce a peptide which is capable of performing the same or similar cytostatic function to that of SEQ ID NO: 1. Examples of such substitutions include: amino acids with aliphatic hydrophobic side chains (Ala, Val, Leu, IIe, Met, Pro, Phe, Trp); amino acids with uncharged but polar side chains (Gly, Ser, Tyr, Asp, Gln, Cys); amino acids with acidic side chains (Asp, Glu); Amino acids with basic side chains (Lys, Arg, His). Conservatively modified peptides which retain the same or similar cytostatic function of the parent peptide are expected to be useful for arresting cell growth and ceasing production of the HIV, in a similar manner to the peptide of SEQ ID NO: 1 or to the peptide corresponding to residues 9-21 of SEQ ID NO: 1.

Accordingly, the scope of protection is not limited by the description set out above, but is only limited by the claims, that scope including all equivalents of the subject matter of the claims. The discussion of a reference in the Description of Related Art is not an admission that it is prior art to the present invention. Each and every original claim is incorporated into the specification as an embodiment of the present invention. Thus the original claims are a further description and are an addition to the preferred embodiments of the present invention. The disclosures of all patents, patent applications and publications cited herein are hereby incorporated herein by reference, to the extent that they provide exemplary, procedural or other details supplementary to those set forth herein.

### REFERENCES

Bagshawe. K. D., Antibody Directed Enzymes Revive Anticancer Prodrugs Concept, Br. J. Cancer 56 (1987) 531-532

Bagshawe, K. D., Springer, C. J. Searle, F., A cytotoxic Agent Can Be Generated Selectively at Cancer Sites. Br. J. Cancer 58 (1988) 700-703

Carpenter, C C J, Fischl, M A, Hammer, S M, et al: Antiretroviral Therapy for HIV Infection in 1996 JAMA 276:146-154, 1996

Doms, R W, Moore, J P. HIV-1 Membrane Fusion: Targets of Opportunity. J Cell Biol 2000; 151: F9-F14.

Ewell, A; Kilmon, J: Federation Proceedings, vol. 40, 1981, Abstract 230.

Ewell, A; Kilmon, J: Federation Proceedings, vol. 40, No. 6 (1981).

Fauci, A S; Pantaleo, G; Stanley, S, etal: Immunopathogenic Mechanisms of HIV Infection. Ann Intern Med 124:654-663, 1996,

Hart, W E, Jr, Swaminathan, S; Beveridge, D I: Molecular Dynamics of HIV-1 Protease. Proteins: Structure, Function and Genetics. 1992 13, 175-194.

Havlir, D V, Richman, D D: Viral Dynamics of HIV: Implications for Drug Development and Therapeutic Strategies. Ann Intern Med 124: 984-994, 1996

Ho, D D, Neumann, A U, Perelson, A S, etal: Rapid Turnover of Plasma Virions and CD4 Lymphocytes in HIV-1 Infection. Nature 373: 123-126, 1995

Kikiwa R, Koyanagi Y, Harada, S, Kobayashi, N, Hazanaga M, Yamamoto, N: Differential Susceptibility to the Acquired Immunodeficiency Syndrome retrovirus in cloned cells of Human Leukemic T-cells line MOLT-4. J. Virol 57:1159-1162 1986

Kilby, J M; Saag, M S: Treatment of HIV-1 Infection: An Overview of New Strategies and Novel Agents. Infect Med 13, 10:903-911, 1996

Laurence, J: The Frustrating Challenge of HIV Therapeutics. The AIDS Reader 5(5):147-150, 1995

Levy, J A (ed) AIDS PATHOGENESIS AND TREATMENT. New York, Marcel Dekker, 1988.

Miller M, Schneider J. Sathyanarayana B K, Toth M V, Marshall G R, Clawson L, Selk L, Kent S B, Wlodawer A. Structure of complex of synthetic HIV-1 protease with a substrate-based inhibitor at 2.3 A resolution. Science 1989 Dec. 1; 246(4934): 1149-52.

Mann, John. Natural products in cancer chemotherapy: past, present and future. Nature Reviews Cancer 2:143-148, 2002

Allen, Theresa M. Ligand-targeted therapeutics in anticancer therapy. Nature Reviews Cancer 2: 750-763, 2002

Raso, V, Griffin T, Hybrid Antibodies with Dual Specificity for the Deliver of Ricin to immunoglobulin bearing target cells. Cancer Res. 1981 41:2073-8

Sattentau, Q J, Moore, J P, The Role of CD4 in HIV Binding and Entry. Phil Trans R. Soc Lond 1993; 342; 59-66

Wei, X, (Ghosh, S K, Taylor, M E, etal: Viral Dynanics in Human lmmunodeficiency Virus type-1 Infection. Nature 373:117-122, 1995

Senter, P D, and Springer C J (2001). Selective activation of anticancer prodrugs by monoclonal antibody-enzyme conjugates. Adv Drug Deliv Rev. 53, 247-64.

McDonagh, C F, Beam, K S, Wu, G J, Chen, J H, Chace, D F, Senter, P D, and Francisco J A (2003) Improved yield and stability of L49-sFv-beta-lactamase, a single-chain antibody fusion protein for anticancer prodrug activation, by protein engineering, Bioconjug Chem, 14, 860-9.

Siemers, N O, Kerr, D E, Yarnold, S, Stebbins, M R, Vrudhula, V M, Hellstrom, I, Hellstrom, K E, and Senter, P D (1997). Construction, expression, and activities of L49-sFv-beta-lactamase, a single-chain antibody fusion protein for anticancer prodrug activation. Bioconjug Chem. 8, 510-9.

Jung, M (2001). Antibody directed enzyme prodrug therapy (ADEPT) and related approaches for anticancer therapy. Mini Rev Med Chem. 1, 399-407.

Wu, A M, and Senter, P D (2005). Arming antibodies: prospects and challenges for immunoconjugates. Nat Biotechnol. 23, 1137-46.

What is claimed is:

1. A method of inducing cytostasis in a population of HIV-infected cells, the method comprising applying to said population of HIV-infected cells a cytostatically effective amount of an inhibitor comprising an isolated peptide having the amino acid sequence of residues 9-21 of SEQ ID NO: 1, or of SEQ ID NO: 1 or a conservative variant thereof, whereby cell growth in at least a portion of said HIV-infected cells is arrested, rendering said growth-arrested cells non-HIV producing.

2. The method of claim 1 wherein applying said peptide to said population of HIV-infected cells further renders at least a portion of said cell growth arrested cells non-proliferating.

3. The method of claim 1 wherein rendering said cell growth arrested cells non-proliferating comprises inducing apoptosis in said cell growth arrested cells.

4. The method of claim 1 wherein said inhibitor comprises said peptide conjugated with a conjugating agent capable of selectively associating the conjugated peptide with an HIV-infected cell, and said step of applying said peptide to said population of HIV-infected cells comprises applying said conjugated peptide.

5. The method of claim 4 further comprising selectively targeting said conjugated peptide to said HIV-infected cell.

6. The method of claim 5, wherein said conjugating agent is selected from the group consisting of polyclonal, monoclonal and bi-specific antibodies, peptides, liposomes, sacchiarides, ligands and receptors, and said step of applying said peptide to said population of HIV-infected cells comprises applying said conjugated peptide.

7. The method of claim 1 wherein said inhibitor is applied to said population of HIV-infected cells in vitro culture.

8. The method of claim 1 wherein said step of applying said peptide to said population of HIV-infected cells comprises selecting a peptide that has at least six cysteine groups and three disulfide bridges.

9. The method of claim 1 wherein said step of applying said peptide to said population of HIV-infected cells comprises selecting a peptide that has a blocked amino terminus.

10. The method of claim 9 wherein said step of applying said peptide to said population of HIV-infected cells comprises selecting a peptide that has a blocked anmino temimius comprising a chemical moiety selected from the group consisting of acetyl, formyl, pyroglutamyl, carbamyl, lactyl, glyceryl and glycosyl.

11. The method of claim 1 wherein said step of applying said peptide to said population of HIV-infected cells comprises selecting a peptide that has an amino acid sequence consisting essentially of no more than about 75 amino acids.

12. A method of treating a patient suffering from HIV/AIDS, comprising:
administering to a patient suffering from HIV/AIDS cytostatically effective amount of an inhibitor comprising an isolated peptide having the amino acid sequence of residues 9-21 of SEQ ID NO: 1, or of SEQ ID NO: 1 or a conservative variant thereof, wherein said inhibitor is selectively targeted to associate with HlV-infected cells in said patient, whereby cell growth in at least a portion of said HIV-infected cells is arrested, rendering said growth-arrested cells non-HIV producing.

13. The method of claim 12 wherein administering said inhibitor to said patient further renders at least a portion of said cell growth arrested cells non-proliferating.

14. The method of claim 12 wherein said step of administering said inhibitor to said patient comprises selecting an inhibitor comprising a peptide having at least six cysteine groups and three disulfide bridges.

15. The method of claim 12 wherein said step of admninistering said inhibitor to said patient comprises selecting an inhibitor comprising a peptide having a blocked amino terminus.

16. The method of claim 12 wherein said step of administering said inhibitor to said patient comprises selecting an inhibitor comprising a peptide conjugated with a conjugating agent capable of selectively associating the conjugated peptide with an HIV-infected cell, and administering said conjugated peptide to said patient.

17. The method of claim 16, further comprising selecting said conjugating agent from the group consisting of polyclonal, monoclonal and bi-specific antibodies, peptides, liposomes, saccharides, ligands and receptors.


SEQUENCE LISTING (5 sequences)

SEQ ID NO: 1 - protein, 39 aa, Crotalus atrox
SQCEQEGGFCRFLLCPSRTSDIGKLGCEPLWKCCKRWGG

SEQ ID NO: 2 - protein, 4 aa, Artificial
  Synthetic
FPNL

SEQ ID NO: 3 - protein, 4 aa, Artificial
  Synthetic
FYNL

SEQ ID NO: 4 - protein, 4 aa, Artificial
  Synthetic
YFNL

SEQ ID NO: 5 - protein, 4 aa, Artificial
  Synthetic
PFNL